SEQUENCE LISTING (8 sequences)

SEQ ID NO: 1 - DNA, 21 bp, synthetic construct
  source 1..21: mol_type = other DNA
cggaaagaaccagacatggac

SEQ ID NO: 2 - DNA, 25 bp, synthetic construct
  source 1..25: mol_type = other DNA
tatcctagggtcacgctatttatgc

SEQ ID NO: 3 - DNA, 22 bp, synthetic construct
  source 1..22: mol_type = other DNA
tatcctaggttcgggggtagtg

SEQ ID NO: 4 - DNA, 19 bp, synthetic construct
  source 1..19: mol_type = other DNA
cctacacccacgcaagacc

SEQ ID NO: 5 - DNA, 24 bp, synthetic construct
  source 1..24: mol_type = other DNA
gctctagatcggggattggtgaac

SEQ ID NO: 6 - DNA, 27 bp, synthetic construct
  source 1..27: mol_type = other DNA
gctctagaatttgttggtaccgaggac

SEQ ID NO: 7 - DNA, 60 bp, synthetic construct
  source 1..60: mol_type = other DNA
catcatcaccaccatcacgagaacctgtacttccagggcagtcacaatgacggcgcaatc

SEQ ID NO: 8 - DNA, 39 bp, synthetic construct
  source 1..39: mol_type = other DNA
gtggcggccgctctattacgcagcgtacatcttgtttac

(19) United States
(12) Patent Application Publication
(10) Pub. No.: US 2025/0295712 A1
(43) Pub. Date: Sep. 25, 2025

(54) COMPOSITIONS AND METHODS FOR MODULATING GUT MICROBIOMES

(71) Applicant: The Regents of the University of Michigan, Ann Arbor, MI (US)

(72) Inventor: Thomas M. Schmidt, Ann Arbor, MI (US)

(21) Appl. No.: 18/836,882

(22) PCT Filed: Feb. 9, 2023
(86) PCT No.: PCT/US2023/062269

§ 371 Date: Aug. 8, 2024

Related U.S. Application Data
(60) Provisional application No. 63/308,338, filed on Feb. 9, 2022.

(51) Int. Cl. A61K 35/745; A61K 31/718

(52) U.S. Cl. CPC A61K 35/745; A61K 31/718

(57) ABSTRACT

Provided herein are compositions and methods for modulating gut microbiomes to treat or prevent diseases. For example, provided herein are microorganisms or components thereof (e.g., proteins involved in associating dietary fibers, such as resistant starch, with beneficial gut microorganisms) that find use treating diseases having an inflammatory component, such as Graft versus Host Disease and cancer. Prebiotic, probiotic, and synbiotic compositions are also provided. In some embodiments, the bacteria are those that naturally express pili that attach to RS granules. In other embodiments, the bacterial are engineered to express or present pili or pilus proteins (e.g., tip pilin) that attach to RS granules.

### CROSS-REFERENCE TO RELATED APPLICATIONS

The present application claims priority to U.S. Provisional Patent Application Ser. No. 63/308,338, filed Feb. 9, 2022, the disclosure of which is herein incorporated by reference in its entirety.

### STATEMENT REGARDING FEDERAL FUNDING

This invention was made with government support under AWD016121 awarded by the National Institutes of Health. The government has certain rights in the invention.

### SEQUENCE LISTING

The text of the computer readable sequence listing filed herewith, titled “40195-601_SEQUENCE_LISTING”, created Feb. 9, 2023, having a file size of 8,158 bytes, is hereby incorporated by reference in its entirety.

### FIELD

Provided herein are compositions and methods for modulating gut microbiomes to treat or prevent diseases. For example, provided herein are microorganisms or components thereof (e.g., proteins involved in associating dietary fibers, such as resistant starch, with beneficial gut microorganisms) that find use treating diseases having an inflammatory component, such as Graft versus Host Disease and cancer. Prebiotic, probiotic, and synbiotic compositions are also provided.

### BACKGROUND

The amount of fiber in a typical Western diet, less than half of the recommended amount³, is a public health concern⁴. In addition to influencing the regularity of bowel movements, fiber contains fermentable compounds that fuel the metabolism of gut microbes. Insufficient dietary fiber translates into decreased output of metabolites from the gut microbiome, many of which promote health⁵. One strategy for addressing the shortcoming in dietary fiber is to supplement diets with purified preparations of plant fiber, such as resistant starch (RS)⁶. Unlike most processed starches, this crystalline form of starch is resistant to digestion by the α-amylases of mammals and most microbes⁷. However, it can be cleaved by specialized bacteria of the gut microbiome that then grow by fermenting the released polysaccharides. These primary degraders of RS also supply cleavage and fermentation products to cross-feed other members of the community⁸ that make other metabolites, some of which impact host health^{9,10}.

Microbes that consume insoluble substrates like RS must first export enzymes that breakdown the RS into maltooligosaccharides or glucose that can be transported into the cell. Releasing diffusible enzymes—‘exoenzymes’—from the cell is an effective strategy for degrading insoluble substrates in stable environments like soil where limited diffusivity is key to driving the evolutionary stability of this strategy¹¹. However, in dynamic environments like the gut, exoenzymes and breakdown products are more likely to be swept away and become ‘public goods’ that benefit multiple microbes in addition to the enzyme-secreting microbes¹². In these turbulent environments, anchoring catabolic enzymes to the cell surface provides microbes with locally enriched concentrations of breakdown products. Indeed, the use of surface-associated enzymes is the strategy employed by prominent members of the human gut microbiota that degrade mucus¹³—an abundant resource in the gut environment. However, for insoluble resources present at lower concentrations, there is selective pressure for extracellular structures that enhance the likelihood of a microbe encountering those substrates. The most studied examples of such extracellular structures in the gut environment are the cellulosomes and amylosomes used by populations of Ruminococcus to capture and degrade cellulose and resistant starch, respectively¹⁴. These extracellular protein complexes comprise substrate binding proteins and enzymes that assemble to collectively encounter, bind and catabolize insoluble substrates. In so doing, cellulosomes and amylosomes effectively increase an organism's fitness by increasing the likelihood of contacting and catabolizing insoluble resources¹⁵. However, there is no genomic or experimental evidence for extracellular machines like cellulosomes or amylosomes in the genus Bifidobacterium—a common member of the human gut microbiome that responds to dietary supplementation with RS^{6,16}.

### SUMMARY

Provided herein are compositions and methods for modulating gut microbiomes to treat or prevent diseases. For example, provided herein are microorganisms or components thereof (e.g., proteins involved in associating dietary fibers, such as resistant starch, with beneficial gut microorganisms) that find use to prevent or treat diseases having an inflammatory component, such as Graft versus Host Disease and cancer. Prebiotic, probiotic, and synbiotic compositions are also provided.

Despite the lack of any recognized mechanism for effectively binding to resistant starch, multiple strains of bifidobacteria grow using RS in vitro^{17,18,19}. One of them (B. adolescentis strain 22L) does so by increasing expression of extracellular, cell wall-associated glycoside hydrolases (GHs) that are anchored to the cell surface¹⁸ as is the case for GHs in many bifidobacteria²⁰.

The technology provided herein takes advantage of the unexpected discovery that bacterial pilus find use in attaching bacteria (e.g., bifidobacterial) to RS granules, promoting RS degradation coupled with bacterial metabolism and growth. Pili in bifidobacteria can be several micrometers in length²¹ and are generally thought to attach bifidobacteria to host cells^{22,23}. As described in the experimental examples below, the microbiome of healthy young adults who supplemented their diets with RSP was analyzed by characterizing metagenomes, bididobateria cultivars, and their pili. Results indicate that pili play an essential role in linking diet to the composition of the gut microbiome.

Thus, in some embodiments, provided herein are synbiotic compositions comprising bacteria expressing pili that attach to RS granules in combination with a resistant starch. In some embodiments, the bacteria are those that naturally express pili that attach to RS granules. In other embodiments, the bacterial are engineered to express or present pili or pilus proteins (e.g., tip pilin) that attach to RS granules. In some embodiments, bacteria are engineered to add or delete one or more genes encoding a factor that affects host health. In some embodiments, probiotic compositions are provided that comprise such bacteria in the absence of resistant starch. In yet other embodiments, prebiotic compositions are provided comprising resistant starch. The compositions may further comprise other components that facilitate research or medical uses.

Also provided herein are methods of using such compositions for therapeutic or research applications. For example, the compositions may be administered to a subject to treat or prevent a disease or condition.

Effective use of RS as a prebiotic to stimulate metabolism of microbial communities in the gut is predicated on the presence of microbes that initiate degradation of this insoluble fiber and provide intermediate degradation products and metabolites to other microbes in the community. The ability to degrade resistant starch has thus far been demonstrated in two families of gut bacteria: the Bifidobactericaeae and the Ruminococcaeceae¹⁷. Ruminococcus bromii L2-63 performs this rare feat using amylosomes, multi-protein complexes similar to the cellulosomes of bacteria that degrade plant cell walls²³. An amylosomes is a complexes of many proteins including polysaccharide cleaving enzymes and structural proteins (scaffoldins) that are assembled by dockerin:cohesin interactions³⁹. Some of the scaffoldins have sortase motifs that anchor them or carbohydrate binding modules (CBMs) for substrate binding. Bifidobacteria use a different system for RS degradation that is based on one or more proteins attached to the cell wall^{19,25}. Each protein can contain one or two catalytic domains and one or more carbohydrate binding motifs (CBMs) that attach to RS—but only when the cells directly encounter RS granules. Provided herein are compositions and methods for longer-range binding of bacteria (e.g., bifidobacteria) to insoluble granules of RS mediated by a starch-binding pilus.

The role of a pilus in binding to RSP was demonstrated multiple ways. Adhesion was lost when the pil^{RS} sortase gene was deleted, and was restored when it was re-introduced. Furthermore, the cloned and heterologously expressed pil^{RS} tip pilin bound raw starch granules and specifically bound amylopectin, the predominant polysaccharide component of starch, via a combination of entropic and enthalpic forces⁴⁰. The Kd for the pilin:amylopectin interaction is on the order of 10⁻⁶ M, which is somewhat tighter binding than that observed for many starch-targeting CBMs. Therefore, this interaction helps bridge longer distances between the bacterium and substrate and provides a high affinity, stabilizing interaction to enhance the efficiency of starch breakdown. It was contemplated that loss of the pilus would reduce the frequency with which RSP granules came into close enough proximity to the RS-degrading enzymes on the surface of bifidobacterial cells⁴¹. Consistent with this, the sortase knock-out strain still degraded RSP—but at a dramatically reduced rate. The parental strain clearly outgrew the knockout strain in head-to-head competition for growth on RSP.

Having concluded that the pilus was responsible for binding to resistant starch and facilitating the catabolism of RSP, initially by strain 269-1 in vitro, we turned to the healthy human cohort to study its potential role in vivo. Metagenome analysis of gut microbiota from RSP-consuming individuals revealed that multiple species of bifidobacteria (B. adolescentis, B. angulatum, and B. pseudo-catenulatum) contain the pil^{RS} locus. Phylogenetic analysis showed that these bifidobacteria are closely related and reside predominantly in one clade—previously designated the “adolescentis” clade³⁶. However, a pil^{RS}-like tip pilin gene was found both in isolates that bind RSP strongly and in those that bind RSP to a lesser extent. These differences in binding suggest that some of these pili may bind primarily to alternative substrates such as other dietary or host-derived polysaccharides. Binding specificity may also explain those metagenomes in our human cohort where there was little to no response during RSP consumption, even though there was a tip pilin homolog present. The diversity and divergence of the tip pilins is consistent with bacteria in the B. adolescentis clade having the ability to bind different substrates and ultimately occupy different niches in the human gut.

Furthermore, the purified pilus tip protein bound tightly to granules of RS and to amylopectin, a major component of RS. Similar tip pilin genes in additional cultivars and metagenomes from our interventional dietary study associated multiple Bifidobacterium species with RSP responses. We found related tip pilin genes in published metagenomes from people world-wide.

In some embodiments, provided herein are synbiotic compositions comprising: a) a resistant starch or other dietary fiber; and b) a bacterium comprising a fiber-binding pilus. In some embodiments, the bacterium is a Bifidobacterium species. In some embodiments, the bacterium is a B. adolescentis clade species. In some embodiments, the bacterium is a B. adolescentis, B. angulatum, B. pseudocatenulatum or B. dentium. In some embodiments, the fiber is composed of resistant starch and is mixed with the bacterium (e.g., to facilitate attachment of the resistant starch to the bacterium). In some embodiments, the resistant starch (e.g., potato starch) is mixed with bacteria prior to the addition of other synbiotic ingredients to the mixture (or the mixture to the other ingredients). In some embodiments, the mixture is incubated for a period of time (e.g., 1 or more minutes, 5 or more minutes, 10 or more minutes, 30 or more minutes, etc.) to facilitate attachment of RS to the bacteria. In some embodiments, the composition comprises a food product, a food, a nutraceutical, or a capsule.

In some embodiments, provided herein are methods comprising: administering a resistant starch and a bacterium comprising a resistant-starch-binding pilus to a subject. In some embodiments, the administering comprising administering a synbiotic composition comprising the resistant starch and the bacterium to the subject (e.g., a mixture of the resistant starch with the bacterium). In some embodiments, the administering comprising administering a probiotic comprising the bacterium to the subject. In some embodiments, the administering comprises administering a prebiotic comprising the resistant starch to the subject. In some embodiments, the administering comprises administering a prebiotic comprising the resistant starch and a probiotic comprising the bacterium to the subject. In some embodiments, the subject has had a bone marrow transplant and so is susceptible to development of graft versus host disease, has cancer, is undergoing cancer a cancer therapy (e.g., treatment with chemotherapeutic agents, checkpoint inhibitors (e.g., type II checkpoint inhibitors), radiation, etc.), has an inflammatory bowel disease, or has another disease or condition.

In some embodiments, initially a prebiotic and probiotic, or synbiotic, are administered to a subject and, after establishment of the bacterium in the gut, continued probiotic is administered to the subject without further need to administer the prebiotic.

### DESCRIPTION OF THE FIGURES

FIG. 1 shows microbiome responses to RSP for the ten ASVs that changed the most. ASVs are arranged with the largest average increase to the left and the largest average decrease to the right. Horizontal bars represent the mean differences in relative abundance for each ASV in those individuals in which it was detected. ASV taxonomic assignments (% nucleotide identity): ASV12—Bifidobacterium adolescentisfaecale stercoris (100%), ASV106 —[Clostridium] leptum (95%), ASV137-Clostridium chartatabidum (99%), ASV7 —[Eubacterium] rectale (100%), ASV22—Prevotella copri (99%), ASV4—Prevotella copri (100%), ASV13-Bacteroides uniformis, ASV63—Bacteroides plebius (100%), ASV116—Eubacterium ruminantium (97%), ASV1—Bacteroides vulgatus (100%).

FIG. 2 shows pilus-dependent binding, degradation, and growth on RSP by B. adolescentis 269-1. Scanning electron micrograph images of a. sterile RS granules, b. RS with B. adolescentis 269-1, c. with B. adolesecentis 269-1 pilin knockout (KO), d. with B. adolescentis 269-1 pilus KO complemented strain (scale bars=2 m), and e. with higher magnification image of B. adolescentis 269-1 bound to RS surface. Scale bar=1 m. f. Model of a typical sortase-dependent pilus in gram-positive bacteria with the assemblage of the three structural pilin proteins attached to the cell wall. g. Growth of WT B. adolescentis 269-1 or the pilin KO strains cocultured on glucose or RSP for 12 h. h. Acetate accumulation from fermentation of RSP: B. adolesecentis 269-1 WT, pilin KO, and pilin KO complemented strains.

FIG. 3 shows purified tip pilin protein bound to amylopectin. a. Predicted domain schematic of Bf1624 using InterProScan²⁷ b. Raw starch binding assay. 8% SDS-PAGE gel showing free protein added, (U) unbound protein in the supernatant, and (B) protein bound to starch granules. c. Example of starch flocculation induced by addition of the tip pilin. d. Affinity electrophoresis gels against 0.3% potato amylopectin, maize amylopectin, glycogen (bovine liver), pullulan, and dextran. BSA as a nonbinding control in each gel to measure band retardation. e. Isothermal titration calorimetry (ITC) results for tip pilin protein (Bf1624) titrating 0.3% potato amylopectin into 25 M protein.

FIG. 4 shows RS-binding ability and tip pilin characteristics of various Bifidobacterium strains: a. Binding of Bifidobacterium strains to RSP in co-sedimentation assays. b. Maximum likelihood phylogenetic reconstruction (bootstrap values above 60% shown) of pilIVRS tip pilin proteins from Bifidobacteria.

FIG. 5 shows relationships between Bifidobacterium populations, genomes and response to RSP consumption. a. Change in reads mapped per million reads (RPM) to Bifidobacterium species after RSP consumption for two weeks. Outlined cells represent metagenomic Bifidobacterium species bins that were assembled in the samples. A “+” indicates the presence of a Pil^{RS} locus in the assembled bin, while a “−” represents its absence in the respective bin. b. Whole-genome maximum-likelihood phylogenetic tree of Bifidobacterium species based on 47 core genes constructed from 100 bootstraps (bootstrap scores ≥70 are shown at the branch points). The clade(s) colored in blue indicate species in which the pilIVRS locus was identified in one or more genome or metagenome. Metagenomic bins and reference genomes are collapsed, the fully expanded genome tree is provided in FIG. 6.

FIG. 6 shows a fully expanded whole-genome maximum-likelihood phylogenetic tree of Bifidobacterium species based on 47 core genes constructed from 100 bootstraps (bootstrap scores ≥70 are shown at the branch points). The clade(s) with lighter color indicate species in which the pilIVRS locus was identified in one or more genome or metagenome.

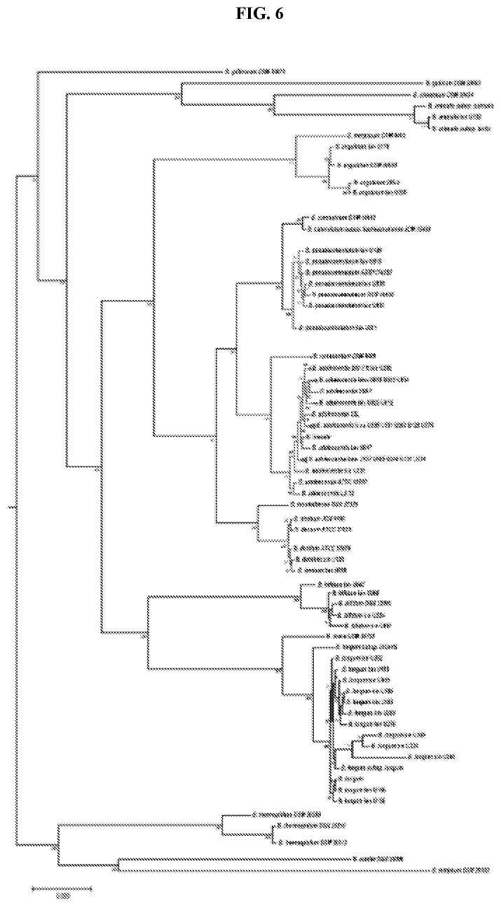

### DEFINITIONS

A “subject” is an animal such as vertebrate, preferably a mammal such as a human or a domestic animal. Mammals are understood to include, but are not limited to, murines, simians, humans, bovines, cervids, equines, porcines, canines, felines etc.

An “effective amount” is an amount sufficient to effect beneficial or desired results. An effective amount can be administered in one or more administrations.

“Co-administration” refers to administration of more than one agent or therapy to a subject. Co-administration may be concurrent or, alternatively, the agents or materials described herein may be administered in advance of or following the administration of the other agent(s) or materials. One skilled in the art can readily determine the appropriate dosage for co-administration.

“Administration” refers to the delivery of one or more agents or therapies to a subject. The present disclosure is not limited to a particular mode of administration. In some embodiments, compositions described herein are delivered orally. In some embodiments, compositions are delivered rectally or through another suitable delivery method.

A “pharmaceutical composition” is intended to include the combination of an active agent with a carrier, inert or active, making the composition suitable for diagnostic or therapeutic use in vivo, in vitro, or ex vivo.

As used herein, the term “pharmaceutically acceptable carrier” encompasses any of the standard pharmaceutical carriers, such as a phosphate buffered saline solution, water, and an emulsion, such as an oil/water or water/oil emulsion, and various types of wetting agents. The compositions also can include stabilizers and preservatives. For examples of carriers, stabilizers and adjuvants see Martin, Remington's Pharmaceutical Sciences, 15th Ed., Mack Publ. Co., Easton, Pa. (1975).

As used herein, the terms “resistant starch” and “RS” refer to any starch that is not digested in the small intestine but passes to the large bowel. “Resistant starch” includes naturally occurring resistant starches and non-resistant starches that are made resistant through a manufacturing process (e.g., by encapsulation, or by chemical modification or other means). The term includes starches that are partially resistant but processed to increase the RS fraction, and processes that produce RS unintentionally (e.g., not engineered specifically to product RS). In some embodiments, RS pass through the intestines completely undigested. In some embodiments, RS are digested very slowly or incompletely.

In some embodiments, RS starches are classified into 5 subtypes, RS1-5 (See e.g., Raigond et al., Journal of the Science of Food and Agriculture, 95:1968 (2015); herein incorporated by reference in its entirety). In some embodiments RS1 comprises starches that are physically inaccessible to digestion (e.g., whole grains or seeds with intact cell walls). In some embodiments, RS2 comprises native starch granules that are protected from digestion by conformation or structure (e.g., raw potatoes or green bananas). In some embodiments, RS3 comprises physical modified starches (e.g., retrograded amylase or other starch such as cooked and cooled potatoes). In some embodiments, RS4 comprises starches that have been chemically modified (e.g., etherized, esterified or cross-bonded with chemicals) to decrease their digestibility. In some embodiments, RS5 comprises RS arising from the formation of amylose-lipid complexes during food process or under controlled conditions.

Examples of RS suitable for use in embodiments of the present disclosure include, but are not limited to, green banana starch, corn starch, potato starch, rice starch, cassava starch, tapioca starch, plantain starch, inulin, or whole or processed foods comprising such RS.

As used herein, the terms “probiotic” and “probiotic compositions” are used interchangeable to refer to live microorganisms (e.g., bacteria) that provide health benefits when consumed or otherwise administered (e.g., orally or rectally).

As used herein, the term “prebiotic” refers to a form of dietary fiber that promotes the growth of beneficial microorganisms in the gut.

As used herein, the term “synbiotic” refers to a mixture, comprising live microorganisms and substrate(s) selectively utilized by host microorganisms, that confers a health benefit on the host. In some embodiments, the synbiotic comprises both a prebiotic and a probiotic.

As used herein, the term “taxon” refers to a group of one or more populations of an organism or organisms that form a unit. A taxon is usually known by a particular name and given a particular ranking, especially if and when it is accepted or becomes established. In some embodiments, taxons are defined by NCBI taxonomy systems. In some embodiments, taxons are defined by the sequence of a marker gene (e.g., a 16S ribosomal RNA sequence). For example, in some embodiments, bacteria 16S sequences that are at least 95% identical to the sequences described herein (e.g., at least 95%, 96%, 97%, 98%, 99%, 99.5%, 99.9%, and fractions thereof) are considered as belonging to the same taxon.

The term “heterologous” as used herein with reference to molecules and in particular enzymes and polynucleotides, indicates molecules that are expressed in an organism other than the organism from which they originated or are found in nature, independently of the level of expression, which can be lower, equal, or higher than the level of expression of the molecule in the native microorganism.

As used herein, the term “heterologous host” refers to a host organism, usually a bacterial strain, that can express one or more genes from another organism (e.g., “source organism”) that is taxonomically classified as belonging to a different genus or species than the host organism. The “heterologous host” has the potential to express a product of the one or more genes from the other organism (e.g., “source organism”) when cultured under appropriate conditions.

### DETAILED DESCRIPTION

In some embodiments, provided herein are compositions and methods comprising a bacterium. In some embodiments, the compositions are probiotic compositions. In some embodiments, the compositions are synbiotic compositions comprising the bacterium and a carbohydrate source (e.g., RS).

In some embodiments, the bacterium has a pilus that binds to resistant-starch. In some such embodiments, the bacterium is a Bifidobacterium species. In some embodiments, the bacterium from the B. adolescentis clade. In some embodiments, the bacterium is a B. adolescentis, B. angulatum, B. pseudocatenulatum or B. dentium. In some embodiments, combinations of two or more different bacterium are provided in the composition.

In some embodiments, the resistant-starch-binding pilus is naturally expressed and presented on the bacterium. In other embodiments, the resistant-starch-binding pilus is heterologously expressed and presented on the bacterium. In some such embodiments, the resistant-starch-binding pilus is derived from one species of Bifidobacterium and expressed in a different species of Bifidobacterium. In other embodiments, the resistant-starch-binding pilus is derived from a specific of Bifidobacterium and expressed in a bacterium that is not a Bifidobacterium species.

The resistant-starch-binding pilus may comprise all or part of a natural or synthetic pilus. In some embodiments, the resistant-starch-binding pilus comprises a tip pilin protein.

In some embodiments, the pilus comprises a heterologous tip pilin protein. In some embodiments, the tip pilin protein is presented on a bacterial surface exposed biomolecule (e.g., protein, lipid, carbohydrate) that is not a pilus.

In some embodiments, the pilus comprises a wild-type amino acid sequence and/or is encoded by wild-type nucleic acid sequences. In other embodiments, the amino acid or nucleic acid sequences may have synthetic, non-natural mutations. Mutations may be employed, for example, to alter binding affinity for a resistant-starch, stability, increased expression, density, localization, or other desired properties.

In some embodiment, prebiotic or synbiotic compositions comprise a carbohydrate. In some embodiments, the carbohydrate source is a resistant starch (e.g., corn, a corn product (e.g. corn starch), potato, green banana starch, a potato product (e.g., potato starch), or inulin).

In some embodiments, the compositions are formulated for oral administration. The present disclosure is not limited to particular methods of oral administration. Examples include, but are not limited to, food products, foods, nutraceuticals, nutritional supplements, capsules, etc.

In some embodiments, the probiotic, prebiotic, and or synbiotic are encapsulated. In some embodiments, a capsule shell that is constructed to dissolve at a predetermined pH of a target region (e.g., large intestine, small intestine, bowel, etc.) is utilized (e.g., available from Assembly Biosciences, Carmel, IN). In some embodiments, capsules also have inner and outer layers that can be engineered to dissolve at different pH levels, making it possible to use a single capsule to deliver two doses of a composition to different locations in the GI tract, or to deliver two different compositions to different locations. In some embodiments, mucin is used in the encapsulation technology to protect against stomach acid.

In some embodiments, the probiotic, prebiotic, and or synbiotic are provided in a single or separate capsules. In some embodiments, the carbohydrate is encapsulated with the bacteria. In some embodiments, the carbohydrate is provided separately. In some embodiments, the carbohydrates is provided as a food or food product and the bacteria is microencapsulated in or on the food or food product. In preferred embodiments, the bacteria and carbohydrate are mixed together to facility attachment of the bacteria to its food source (e.g., resistant starch).

In some embodiments, the carbohydrate and/or the bacteria are formulated for rectal administration (e.g., as a suppository).

In some embodiments, compositions are formulated in a pharmaceutical composition. The bacteria of embodiments of the disclosure may be administered alone or in combination with pharmaceutically acceptable carriers or diluents, and such administration may be carried out in single or multiple doses.

Compositions may, for example, be in the form of tablets, resolvable tablets, capsules, bolus, drench, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the disclosure may be in a form suitable for oral or rectal administration. Depending upon the disorder and patient to be treated and the route of administration, the compositions may be administered at varying doses.

For oral administration, bacteria of embodiments of the present disclosure may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Other suitable fillers, binders, disintegrants, lubricants and additional excipients are well known to a person skilled in the art. In some embodiments, compositions include one or more of mucin, antioxidants, reductants, or redox-active compound (e.g., to protect bacteria).

In some embodiments, bacteria are spray-dried. In other embodiments, bacteria resuspended in an oil phase and are encased by at least one protective layer, which is water-soluble (water-soluble derivatives of cellulose or starch, gums or pectins; See e.g., EP0180743, herein incorporated by reference in its entirety).

In some embodiments, compositions are provided as a nutritional or dietary supplement. In some embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The dietary supplement may comprise one or more inert ingredients, especially if it is desirable to limit the number of calories added to the diet by the dietary supplement. For example, the dietary supplement may also contain optional ingredients including, for example, herbs, vitamins, minerals, enhancers, colorants, sweeteners, flavorants, inert ingredients, and the like. For example, the dietary supplement may contain one or more of the following: asorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, compositions are provided as nutritional supplements (e.g., energy bars or meal replacement bars or beverages). The nutritional supplement may serve as meal or snack replacement and generally provide nutrient calories. In some embodiments, the nutritional supplements provide carbohydrates, proteins, and fats in balanced amounts.

Sources of protein to be incorporated into the nutritional supplement are any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. Guidance to the amounts or ingredients can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In some embodiments, the nutritional supplement contains natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In one embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

In some embodiments, compositions are provided as food products, prepared food products, or foodstuffs comprising carbohydrate and bacteria as described above. For example, in some embodiments, beverages and solid or semi-solid foods are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

In some embodiments, compositions described herein are administered one or more times (e.g., daily, multiple times a day, multiple times a week, multiple times a month, etc.) for a period of time (e.g., weeks, months, years, indefinitely). In some embodiments, compositions are administered in order to obtain and maintain a desired outcome (e.g., reduction in symptoms of a disease or condition, etc.).

In some embodiments, one or more biomarker levels of an individual are assayed one or more times prior to or during treatment. In some embodiments, the treatment is altered (e.g., change in dosage or specific bacteria and carbohydrate administered) in response to the presence of, absence of, or level of biomarker. For example, if a biomarker level indicative of continued disease or poor gut microorganism levels or metabolic activity remain below, the amount or type of bacteria and/or carbohydrate can be altered. In some embodiments, the levels of biomarker are then assayed again to assess the new treatment.

In some embodiments, the present disclosure provides kits, pharmaceutical compositions, or other delivery systems for use in analyzing or treating a disease or condition in an animal and/or identifying animals in need of treatment with the compositions and methods described herein. The kit may include any and all components necessary, useful or sufficient for research or therapeutic uses including, but not limited to, one or more bacteria, carbohydrate sources, pharmaceutical carriers, PCR primers, reagents, enzymes, buffer, and additional components useful, necessary or sufficient for treating an animal or identifying animals in need of treatment with the compositions described herein. In some embodiments, kits include directions for performing diagnostic assays and/or determining a treatment course of action based on the results of the diagnostic assay. In some embodiments, the kits provide a sub-set of the required components, wherein it is expected that the user will supply the remaining components. In some embodiments, the kits comprise two or more separate containers wherein each container houses a subset of the components to be delivered.

Optionally, compositions and kits comprise other active components in order to achieve desired therapeutic effects and/or perform diagnostic assays.

The compositions and methods provided herein find use in the treatment and/or prevention of diseases and conditions including, but not limited to, graft versus host disease (GvHD) (e.g. associated with bone marrow transplant), cancer (e.g., colorectal cancer), inflammatory bowel diseases (e.g., ulcerative colitis, Crohn's disease), irritable bowel syndrome, obesity, diabetes (e.g., type II diabetes), leaky gut, metabolic syndromes, neurological disorders, conditions, diseases, and disorders caused by infectious disease agents (e.g., Lyme disease), and liver diseases (e.g., nonalcoholic steatohepatitis (NASH), cirrhosis, hepatic encephalopathy), and for addressing symptoms including, but not limited to, inflammation, bloating, abdominal pain, diarrhea, and poor sleep.

Any suitable dose of probiotic, prebiotic, or synbiotic may be used with any particular subject. In some embodiments, the prebiotic may be provided in a range of 10 to 40 grams a day (e.g., 20 grams a day) for a human subject, although higher or lower amounts may be given. In some embodiments, the probiotic bacterium is provided in a range of 1 to 10 billion colony forming units per dose (e.g., daily dose), although higher or lower amounts may be given.

### EXAMPLES

### Methods

### Human Cohort Participants and Study Design.

Various commercially available dietary fibers were used between Winter 2015 and Winter 2020. Data herein are from individuals who supplemented their diet twice daily with approximately 20 grams of resistant starch product prepared from potatoes (RSP) by Bob's Red Mill, Milwaukee, WI or LODAAT Pharmaceuticals, Regis, IL. The RSP from both suppliers contained approximately 65% w/w resistant starch as measured by AOAC 2002.02 1.

Participants self-collected 3-4 fecal samples using the Omnigene Gut (DNA Genotek) during the first week of the study which is prior to consuming the RSP fiber supplement. No samples were collected during the second week of the study during which participants consumed a single 20-gram dose of RSP for two days, followed by consumption of two doses per day for five days. During the third week of the study, participants continued consuming RSP twice per day and collected 3-4 additional fecal samples. Only data from individuals consuming more than 75% of the prescribed doses of RSP, based on self-recorded logs, were used in this study. Aside from the supplement, participants maintained their normal diet throughout the study.

16S rRNA Gene Sequencing and Analysis.

DNA was extracted from 250 L of fecal suspension using the 96-well MagAttract PowerMicrobiome DNA isolation kit (Qiagen) and EpMotion liquid handling systems (Eppendorf). The V4 region of the bacterial 16S rRNA gene was amplified and sequenced as described previously² using 2×250-bp paired-end kits on the Illumina MiSeq platform. The samples were distributed randomly for DNA extraction and sequencing. Sequences were curated using the mothur software package^{2,3}. Unless stated otherwise, species designations indicate 100% identity to a single species in the database.

To identify Amplicon Sequence Variants (ASVs) that changed most frequently and to the largest extent in response to RSP, we first identified ASVs that were detected at any time in at least 10% of the individuals who provided at least two samples from week one (normal diet) and at least two samples from week three (supplemented diet) (140 participants). We then calculated the differences between the averages for week one and week three were then determined for each ASV in each person. The ten most responsive ASVs had the largest positive or negative average differences Bifidobacterium isolation and growth.

Fecal sample from four individuals were serially diluted and plated onto Bifidus Selective Medium agar (BSM-Agar; Sigma-Aldrich) plates including the BSM supplement as per the manufacturer's recommendation. The plates were incubated in a COY anaerobic chamber containing ca. 92% nitrogen, 3% hydrogen, and 5% carbon dioxide for one week. Bifidobacterium colonies developed a dark pink center and light brown edge. Clearly isolated colonies were picked and streaked on BSM plates for further purification. The taxonomic identity of isolates was determined from the amplified (primers 8F (5′-AGAGTTTGATCCTGGCTCAG3′ (SEQ ID NO:1)) and 1492R (5′-GGTTACCTTGTTACGACTT-3′ (SEQ ID NO:2))) 16S rRNA gene. Strains identified as Bifidobacterium (greater than 99% identity to previously described bifidobacteria in NCBI's database of 16S ribosomal RNA sequences) were grown to exponential growth phase in BSM broth. Five percent DMSO was added to culture aliquots that were subsequently stored at −80° C. All strains were routinely grown in 10 mL anaerobic YCFC medium which is a slight modification of YCFA developed by Harry Flint and colleagues⁴. The major change is a reduction in bicarbonate to 1 g/l to be compatible with different CO₂ concentrations and the addition of 100 mM MOPS to help neutralize fermentation products and extend growth. The medium was supplemented with either glucose (22 mM) or RSP (1% w/v) as indicated.

### Genome Sequencing and Analysis.

Total DNA from B. adolescentis strain 269-1 was sequenced using both PacBio and Illumina MiSeq sequencing platforms. For PacBio sequencing, the DNA was prepared using Genomic DNA kit (Qiagen). For Illumina sequencing, DNA was prepared from 1.5 mL overnight cultures using Qiagen's DNeasy PowerSoil Pro Kit. Two other Bifidobacterium strains were sequenced using Illumina MiSeq alone: B. pseudocatenulatum strain 301-3, and B. angulatum strain 285-2. Genomic MiSeq reads were assembled with SPAdes5 v.3.13.0 and annotated with RASTtk6 v1.073 using the KBase7 workflow (narrative.kbase.us/narrative/48493). The hybrid PacBio+Illumina assembly was produced using (Unicycler 8 v0.4.8 or hybridSPAdes 9 (v3.13.0) and assemblies were annotated with RAST-tk in the same way as the short-read Illumina genomes. To identify the pil^{RS} loci in these isolate genomes, the RAST annotations were manually searched for the presence of a “LPXTG specific Sortase A”, a “collagen adhesin precursor” (backbone pilin), a “cell wall surface anchor family protein” (basal pilin), and “hypothetical protein” of around ˜1250-˜1450 amino acids long (tip pilin); all pil^{RS} proteins were required to be located in the conserved genome context as observed in the B. adolescentis strain 22L10 (specifically, Tip-Basal-Backbone-Sortase). Reasonable protein Identity (>%30 ID) was confirmed for each homolog with BLASTp11. Reference genome assemblies for B. adolescentis strains 22L and L2-32 were downloaded from NCBI and annotated with the same pipeline; genome assemblies of B. adolescentis ATCC 15703, B. dentium ATCC 27678, B. bifidum ATCC 29521, and B. longum ATCC 15697 were obtain from the ATCC and then analyzed with the pipeline as described above. Tip pilin protein sequences from strains possessing the pil^{RS} locus were aligned using CLUSTAL W12 with the BLOSSUM62 matrix and default scoring parameters in the MEGA713 software package. MEGA7 was also used to construct a phylogenetic tree using the Maximum Likelihood method based on the Poisson correction model14 with 100 bootstraps. Domain architecture searches were performed with InterProScan15 v5.52 webservice (ebi.ac.uk/interpro/search/sequence/) and visualized with the Illustrator for Biological Sequences (IBS) software¹⁶.

### Transcriptomic Analysis.

Total RNA was extracted from 2 ml of mid-exponential phase cultures of B. adolescentis strain 269-1 in YCFC medium supplemented with either glucose or RSP as the primary carbon source. RNA was stabilized before extraction using RNAprotect Bacteria Reagent (Qiagen) in a 1:1 ratio, and the mixture was pelleted by centrifugation at 3,000 g for 15 min at 4° C. The RNA was then extracted using the RNeasy PowerMicrobiome Kit (Qiagen) according to the manufacturer's instructions with an on-column DNase treatment. The extracted RNA was stored in 50 μL of water at −80° C. Library preparation and Illumina Sequencing were performed by the University of Michigan Microbiome Core. Trimmed and filtered mRNA reads from triplicate samples for each of the two different culture conditions were mapped against the B. adolescentis strain 269-1 genome (genebank/NCBI id?). All steps for analyzing computational tools were executed within a single analytical workflow (github.com/jgolob/transcriptshot).

### Construction of a Sortase-Deleted Mutant.

The sortase gene homologous to the pil4 sortase in B. adolescentis strain 22L was deleted in B. adolescentis strain 269-1. The sequences of all primer pairs used for the construction of deletion strain and its complement are listed in Table 4.

TABLE 4
Primers used in construction of sortase deletion mutant and for heterologous
expression of recombinant tip protein.

| Primer Name | Primer Sequence^{a} |
|---|---|
| Up-f | CGGAAAGAACCAGACATGGAC (SEQ ID NO: 1) |
| Up-r | TATCCTAGGGTCACGCTATTTATGC (SEQ ID NO: 2) |
| Dn-f | TATCCTAGGTTCGGGGGTAGTG (SEQ ID NO: 3) |
| Dn-r | CCTACACCCACGCAAGACC (SEQ ID NO: 4) |
| ermG-f | GCTCTAGATCGGGGATTGGTGAAC (SEQ ID NO: 5) |
| ermG-r | GCTCTAGAATTTGTTGGTACCGAGGAC (SEQ ID NO: 6) |
| N-HPL_61-F | catcatcaccaccatcacGAGAACCTGTACTTCCAGGGCAGTCACAATGACGGC |
| | GCAATC (SEQ ID NO: 7) |
| N-HPL 1301-R | GTGGCGGCCGCTCTATTACGCAGCGTACATCTTGTTTAC (SEQ ID |
| | NO: 8) |

^{a}Bolded letters denote added restriction sites; lowercase letters represent the introduced His-tag with TEV cleavage site.

Primer pairs were designed to amplify regions approximately 500 bp upstream and downstream of the target genes and to add AvrII (CCTAGG) restriction sites to the PCR products. These products were ligated into the pCR2.1 TOPO cloning vector. The erythromycin cassette was amplified from pNBU2-bla-ermGb plasmid¹⁷ and inserted into the middle of upstream and downstream plasmid. Plasmid carrying the deleted sortase gene was linearized by digestion with XbaI and transformed into B. adolescentis strain 269-1 cells by electroporation¹⁸. Isolated colonies were grown on YCFC agar plates supplemented with erythromycin (2 g/mL). The resulting strain with an erythromycin resistance cassette replacing the sortase gene was designated A1627.

The sortase deletion was complemented by transformation of strain A1627 with a plasmid carrying a fragment from approximately 500 bp upstream of the sortase gene to approximately 500 bp downstream position. This plasmid was introduced into strain A1627 by electroporation. Colonies were grown on YCFC plates without antibiotic and screened side by YCFC plates with antibiotic. The complemented strain was designated A1627 complementary strain.

### Adhesion to Starch Granules.

Adhesion to starch granules was measured by a co-sedimentation assay¹⁹. Cells harvested from stationary phase culture on YCFC supplemented with glucose, were washed twice with 0.1 M phosphate buffer (pH 7.0) before they were resuspended in the same buffer at an OD540 of approximately 0.07. RPS was suspended in 0.1 M phosphate buffer (pH 7.0) at concentration of 0.5 g mL⁻¹. Equal volumes of bacteria and starch suspensions were mixed thoroughly and incubated at room temperature for 1 h. Controls contained bacteria but no starch or starch but no bacteria. The concentration of non-adhered cells was determined by the OD540 of samples taken from 0.5 cm below the liquid surface. Strains in which more than 70% of the cells adhered to the starch granules were considered highly adherent. Less than 40% adhesion was considered poor.

### Scanning Electron Microscopy.

After 6 h incubation in YCFC medium supplemented with RSP, cultures were fixed on 13 mm diameter Thermanox coverslips with 2% glutaraldehyde (GA) in 0.1 M cacodylate buffer pH 7.2 (CB) at 4° C. overnight. Samples were washed 3 times in 0.1 M CB pH 7.2, 2 min each at room temperature. Conductive layers were prepared at room temperature. The first conductive layer was stained with osmium tetroxide (2% OsO4 in 0.1 M CB) for 5 min, then washed 3× with 0.1 M CB, followed by 2% tannic acid in 0.1 M CB for 5 min, and washed with 0.1 M CB. The second conductive layer was stained in the same way as the first. The third conductive layer was stained only with only 2% OsO₄ in 0.1 M CB. Dehydration was achieved with ice-cold ethanol of increasing concentrations: 30%, 50%, 70%, 80%, 90%, 95%, and twice with 100%, for 5 min each. Samples were dried with hexamethyldisilazane (HAMIDS) for 10 min twice then evaporated at room temperature overnight. Dried samples were sputter coated with Au (20 nm thick) using a Leica EM ACE600 high vacuum coater (Leica Microsystems Inc., USA). Observations were made by a Carl ZEISS EVO 15 LaB6 scanning electron microscope (ZEISS Inc., USA) and TESCAN MIRA3 scanning electron microscope with a field emission gun (FEG) (TESCAN Inc., USA).

### Co-Culture Experiment.

B. adolescentis strain 269-1 and B. adolescentis strain A1627 were cultured in YCFC medium with glucose at 37° C. When the OD600 reached 0.8, equal volumes of cells were mixed thoroughly and inoculated into YCFC medium supplemented with glucose or RS. After 12 h incubation, cultures were spread on YCFC agar plates with or without erythromycin to determine the concentration of colony forming units (CFU) of the deletion mutant or of both strains, respectively.

### Fermentation in Buffer.

B. adolescentis strains 269-1, A1627 and its complement were each cultured in 50 mL YCFC medium supplemented with glucose at 37° C. overnight. Cells were washed three times with 0.1 M phosphate buffer (pH 7.0) to remove residual medium and then resuspended in 10 ml 0.1 M phosphate buffer (pH 7.0) and 10 g·L⁻¹ RPS. All tubes were incubated in a 37° C. shaker (Thermo Scientific MaxQ 8000, 150 rpm) and sampled periodically. Accumulation of the fermentation product acetate was measured in a Shimadzu IPLC system (Shimadzu Scientific Instruments, MD) with an Aminex HPX-87H column (Bio-Rad Laboratories, CA). The sample was eluted by 0.01N H₂SO₄ at a total flow rate of 0.6 mL per min with the column oven temperature at 50° C.

### Cloning and Recombinant Protein Expression.

The full-length tip pilin having amino acids 61-1301 sans N-terminal signal peptide sequence and C-terminal predicted transmembrane helix was cloned using the commercial Lucigen Expresso T7 Cloning & Expression System (cat #49001-2) as previously described²⁰. The N-terminal primers (Table 4) introduced a TEV-cleavable site between the mature protein and the His tag to produce soluble TEV-cleavable His-tagged proteins. Recombined pETite plasmid was transformed into Rosetta (DE3) pLysS cells (EMD Biosciences) and plated on LB agar containing 50 g mL⁻¹ kanamycin (Kan) and 20 g mL⁻¹ chloramphenicol (Cm). After 16 h of growth at 37° C., colonies from plates were used to inoculate 1 liter of terrific broth with Kan and Cm for growth at 37° C. When culture OD600 reached about 0.6, 0.5 mM IPTG was added and cells were incubated at room temperature overnight. Cells were harvested by centrifugation and stored at −80° C. until purification.

### Recombinant Protein Purification.

The cell pellet was resuspended in 50 mL of Talon Buffer (20 mM Tris, 300 mM NaCl, pH8.0) and lysed by sonication. Lysates were centrifuged at 30,000×g at 4° C. and the supernatant loaded onto a 5 ml HisPur Cobalt resin (Thermo Fisher Scientific, MA) equilibrated with Talon Buffer. Protein was eluted with an imidazole (5-500 mM) gradient. The His-tag was removed by a 2 h incubation with recombinant TEV (1:100 molar ratio of TEV to protein) at room temperature, followed by an overnight incubation at 4° C. while dialyzing into Talon buffer. Affinity purification was repeated to remove the His-tag, uncut protein and His-tagged TEV. The cleaved protein was then dialyzed against a storage buffer (20 mM HEPES, 100 mM NaCl, pH 7.0) at 4° C. and concentrated using a Vivaspin 15 (10,000 MWCO) centrifugal concentrator (Vivaproducts Inc., MA).

### Affinity Gel Electrophoresis.

Continuous native polyacrylamide gels were prepared consisting of 8% (w/v) acrylamide in 37.5 mm Tris-HCl pH 8.8, TEMED and ammonium persulfate (APS) to initiate polymerization. Amylopectin (0.3% (w/v) was added to one of the gels prior to polymerization. Approximately 5 g of tip pilin protein or bovine serum albumin (as a noninteracting negative control) were loaded on the gels and electrophoresis was carried out for 3 h in an ice bath. Gels were stained with Coomassie Brilliant Blue.

### Tip Pilin Starch Binding Assays.

To determine the ability of the recombinant tip pilin to bind raw potato (Bob's Red Mill), corn (Sigma #S4126), or wheat (Sigma #S5127) we washed each starch 3× with protein buffer (20 mM HEPES, 100 mM NaCl) to remove free starch. The starch and 10 μM protein were mixed end-over-end for 1 hour at room temperature. Starch was then pelleted at low speed to separate bound and free protein. Qualitative judgments of starch binding were made by adding urea to denature protein bound to starch granules in the same volume as the original reaction and then running the starch-bound and free protein fractions on a denaturing SDS-PAGE gel. To determine the protein's ability to bind various polysaccharides, we performed affinity electrophoresis as previously described²¹. Briefly, non-denaturing acrylamide gels were prepared with poylsaccharides in the gel matrix. Gels were run at low voltage on ice for 4 hours and binding evaluation was made by measuring the distance between the protein band and the stacking gel and comparing it to the non-polysaccharide gel. Binding was considered positive if the ratio was <0.85.

### Isothermal Titration Calorimetry (ITC).

Isothermal titration calorimetry was performed to determine binding parameters as described previously for soluble polysaccharides²¹. Experiments were performed in duplicate by titrating 0.3% sterilized potato amylopectin into 25 M protein on a TA Instruments large volume NanoITC and modeled using TA Instruments' NanoAnalyze software. The input substrate molarity was adjusted until n=1, assuming binding of the protein as one whole binding site, and determining from that the dissociation constant (KD) and the concentration of binding sites over the w/v % of amylopectin.

### Metagenome Sequencing, Assembly, Annotation, and Taxonomy, and Read Mapping.

DNA was extracted from fecal samples using the MagAttract PowerMicrobiome kit (Qiagen). Metagenomic DNA was prepared for sequencing on a Novoseq (Illumina) using the Ultra II FS DNA Library Prep Kit (NEB, E7805). Similar to isolate genomes, the KBase pipeline⁷ was used to assemble and annotate metagenomes according to the following workflow. First, paired-end reads were merged and trimmed with Trimmomatic²² v0.36 with leading and tailing minimum quality and a minimum read length of at least 36 bp. Next, reads were assembled with metaSPAdes²³ v3.13.0 or IDBA-UD²⁴ v1.1.3 in the case a sample encountered a metaSPAdes run time error; minimum contig lengths were set at 2.0 kb by default and other default parameters of the KBase pipeline were used. The assembled contigs were then simultaneously binned using MaxBin2²⁵ v2.2.4, CONCOCT²⁶ v1.1, and MetaBAT2²⁷ v1.7 which were then optimized and dereplicated into one final set of assembled metagenomes using the DAS-Tool²⁸ v1.2. Final assemblies were assessed with CheckM²⁹ v1.0.18 and filtered to keep genomes that were at least 90% complete and contained less than 5% contamination. Finally, assemblies were then annotated with RASTtk^{6,30,31} v1.073 for downstream analysis. Bifidobacterium bins were identified with the GTDB-Tk³² v1.0 toolkit and then searched for intact full pilIVRS loci as described above in the “Genome sequencing and analysis” section. Using the SpeciesTree app in Kbase, homologs of 49 core universal genes were aligned and used to reconstruct the phylogenetic relationships of the Bifidobacterium metagenomic bins with FastTree2³³. To assess the RPS-related changes in the Bifidobacterial population changes at the metagenome level, metagenomic reads were binned by mapping them with BBSplit (sourceforge.net/projects/bbmap/) versus the genomes of B. adolescentis ATCC 15703, B. dentium ATCC 27678, B. bifidum ATCC 29521, B. longum ATCC 15697, B. animalis subsp. Lactis ATCC 700541, B. angulatum DSM 20098, B. pseudocatenulatum DSM 20438. The reads mapped per million reads (RPM) were calculated from number of reads mapped to each genome divided by the number of total reads for each sample; the RPM at start of RPS supplementation was subtracted from the RPM at end of RPS supplementation.

### Example 1

### Microbiome Responses to RSP Consumption

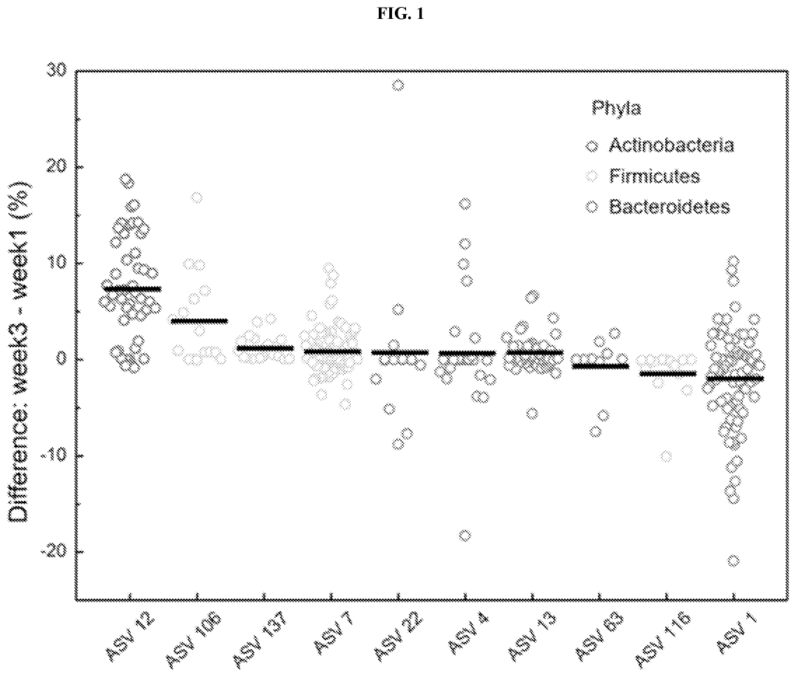

In a cohort of 140 healthy young adults who supplemented their diets with resistant starch from potatoes (RSP), the largest and most common response in the gut microbiome was an increase in the relative abundance of bacterial populations with a V4 16S ribosomal RNA sequence identical to B. adolescentisfaecale stercoris (ASV12; FIG. 1). ASV12 was not detected in everyone and the magnitude of the response differed between individuals.

To identify characteristics that support blooms of ASV12, we isolated bifidobacteria from multiple study participants. Strain 269-1 was isolated from a participant in whom ASV12 increased 14.5-fold in response to RSP. It has a V4 region identical to ASV12 and was selected for more detailed study. Whole genome analysis revealed 98% average nucleotide identity (ANI) between strain 269-1 and the type strain of B. adolescentis (strain E194a, ATCC15703) over 82% of their genomes. This degree of similarity falls within the guidelines²⁴ for designating strain 269-1 as a member of the species B. adolescentis. The genome also contains homologs of genes that were transcriptionally induced by RS in B. adolescentis strain 22L18. Among them are four genes that are syntenic and code for the three structural proteins of a Type IVa pilus, along with the sortase that covalently cross-links the structural proteins.

### Example 2

### Starch Binding by a Type IVa Pilus

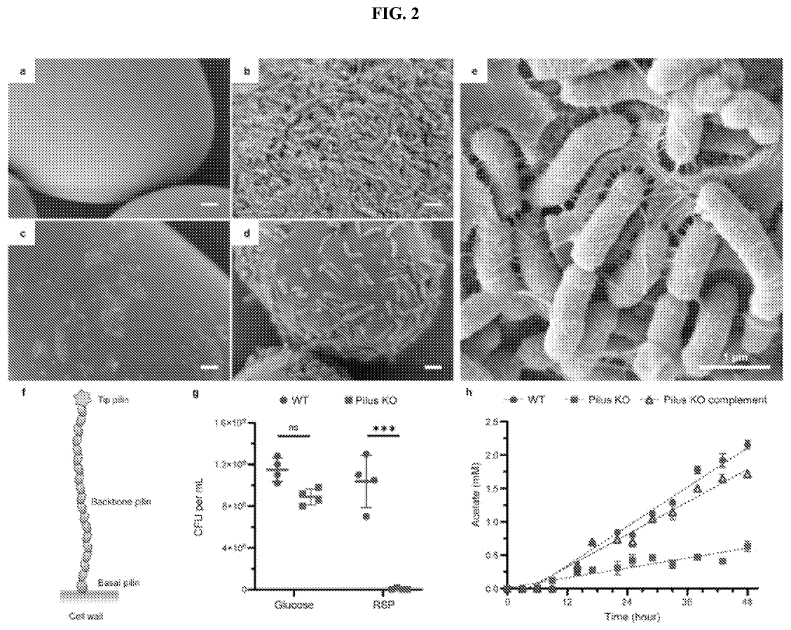

The observation that only one of the five Type IVa pili in strain 22L was induced during growth on RS suggested to us that this pilus is involved primarily in binding RSP, rather than host cells as originally suggested¹⁸. To test this hypothesis, the sortase gene in the pil locus of strain 269-1 was deleted and then complemented through recombineering²⁵. Microscopic examination revealed that, while cells of the parental strain quickly adhered to the surface of RSP granules (FIGS. 2a and 2b), the sortase deletion mutant did not (FIG. 2c). Binding to RSP was restored when the sortase gene was reinserted into the genome (FIG. 2d). Pili are visible in the parental strain at high magnification (FIG. 2e) and its genome contains genes for the structural proteins of a Type IVa pilus (FIG. 2f). The loss of adhesion did not completely prevent fermentation of RSP by the sortase knock-out, but the rate was dramatically reduced (FIG. 2g). The knock-out essentially rendered the strain non-competitive against the wild-type for growth on RSP in mixed cultures (FIG. 2h). With this demonstration of a pilus involvement in binding RS, we designated its genomic locus as pil^{RS}.

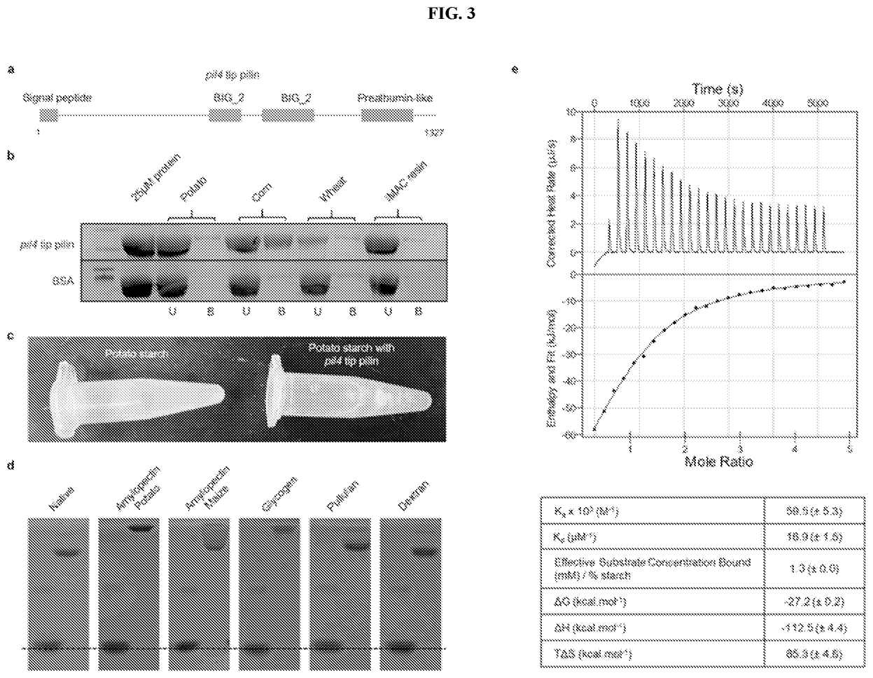

To determine if the pil^{RS} tip pilin is capable of binding starch, we expressed a construct of amino acids 61-1301 (lacking the predicted signal peptide and C-terminal transmembrane helix) as a soluble protein in E. coli. Bioinformatic analysis via dbCAN26 and InterPro Scan27 failed to identify any known carbohydrate binding motifs in the tip pilin (FIG. 3a) and so it is likely to harbor novel starch-binding domains. The tip pilin bound to raw starch from potato, maize, and wheat despite their differences in crystallinity type, proportion of amylose to amylopectin, size, phosphorous form²⁸, and accessibility to amylases²⁹. Most notably, the addition of the tip pilin immediately induced starch flocculation (FIG. 3c) suggesting that the tip pilin alone is sufficient to induce flocculation of raw starch observed in bacterial cultures³⁰,3¹,3². The rate of migration of the tip protein through non-denaturing gels supplemented with different polysaccharides was used to determine which α-glucan polysaccharides bind the tip pilin. We found that the tip pilin bound to potato amylopectin, maize amylopectin, glycogen, and pullulan (FIG. 3d). The difference in migration suggests that the tip pilin binds more tightly to potato than maize amylopectin. Using isothermal titration calorimetry (ITC) with the number of binding sites fixed at one³³, binding to potato starch was the strongest (1.3 mM per % w/v of potato amylopectin) with an effective dissociation constant (Kd) of 16.9+/−1.5 μM. This affinity is on the same order of magnitude as CBM21 for amylose34 and pairs of starch binding CBMs from Amy13K from Eubacterium rectale for glycogen³⁵.

Although the pil^{RS} locus in strain 269-1 is clearly involved in binding to RS, pilus production was not induced by RS as it is in strain 22L. Transcript levels of pil^{RS} genes were similar in strain 269-1 grown with glucose or RPS as the primary carbon source, when induction of potential RS-degrading enzymes was readily detectable (Table 1). This difference in inducibility may result from different numbers of Type IVa pil loci in the two strains (five in 22L but only one in 269-1). Constitutive expression of all Type IVa pil loci would demand more sources in 22L than it would in 269-1 so regulated expression in 22L may confer a selective advantage.

TABLE 1
Expression of pil^{RS} genes in B. adolescentis strain 269-1 grown
in glucose or Resistant Potato Starch. Transcripts were assigned
to coding sequences then normalized to gene size and sample size.
The resulting Fragments Per Kilobase of Gene per Million total
reads (FPKM) are shown as the average of three replicates ±
the standard error of the mean. The positive controls for induction
by RPS are two genes that code for extracellular amylases believed
to be involved in the initial cleavage of RPS granules.

| | | Glucose | RPS | |
|---|---|---|---|---|
| Gene | Function | (FPKM) | (FPKM) | Induction |
| cds_001716 | Sortase | 132 ± 16 | 214 ± 82 | 1.6 |
| cds_001717 | Backbone Pilin | 174 ± 31 | 214 ± 81 | 1.2 |
| cds_001718 | Base Pilin | 1256 ± 451 | 844 ± 178 | 0.7 |
| cds_001719 | Tip Pilin | 246 ± 41 | 647 ± 251 | 2.7 |
| cds_000713 | Amylopullulanase | 96 ± 38 | 2187 ± 355 | 24 |
| cds_001505 | Amylase | 121 ± 54 | 2066 ± 777 | 17 |

### Example 3

### The Pil^{RS} Locus in Other Bifidobacteria

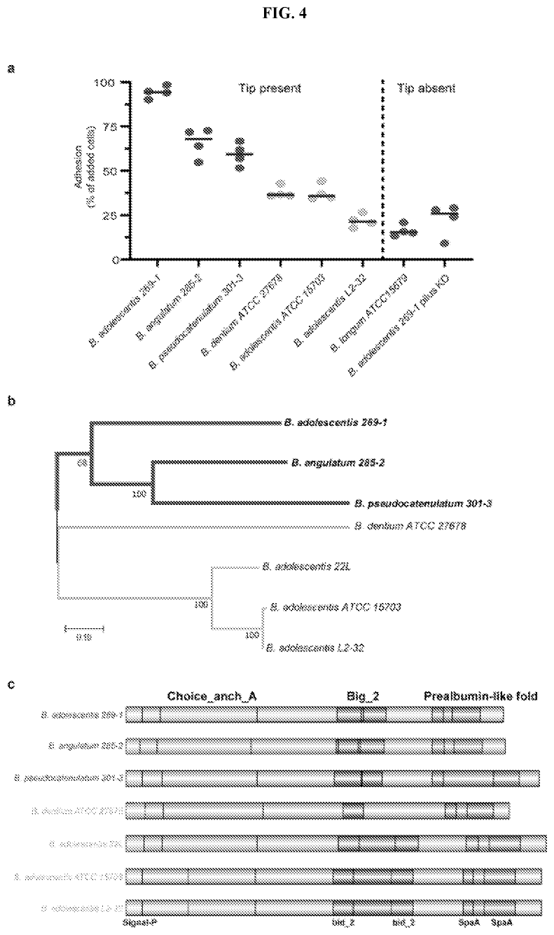

Given the importance of pil^{RS} for RS utilization by B. adolescentis strain 269-1, we looked for similar genes and RSP-binding in other cultivars of Bifidobacterium. RSP adherence was determined by mixing Bifidobacterium cultures with RSP and monitoring the optical density of the supernatant fraction after the starch granules settled. In addition to B. adolescentis (269-1), co-sedimentation with RSP granules was observed with cultivars of B. angulatum (285-2) and B. pseudocatenulatum (301-3; FIG. 4a). There was modest cosedimentation with strains of B. dentium (ATCC27678) and two other strains of B. adolescentis (L2-32 and 299-1). There was no measurable co-sedimentation with a strain of B. longum (ATCC 15697) or the sortase knockout strain of B. adolescentis 269-1. The affinity of strains for binding RSP was reflected in the phylogeny of the tip pilin (FIG. 4b), with the strongest RSP binders contained in a clade distinct from the weakly binding strains. Each tip pilin protein sequence is comprised of a similar domain topology (FIG. 4c). The tip pilin sequence of B. adolescentis (269-1) shares ˜40% identity with the other pilin proteins, suggesting that most of the structure is conserved. Perhaps the most striking difference is the insertion of ˜120 amino acids within the predicted bacterial immunoglobulin (BIg_2) domain for three of the moderately-binding strains, and a shortened BIg_2 for the B. dentium strain. Because many bacterial glycan-binding proteins and adhesins are comprised of multiple Ig-like domains to enhance avidity, it is possible the insertion alters the positioning of starch-binding motifs, weakening affinity.

### Example 4

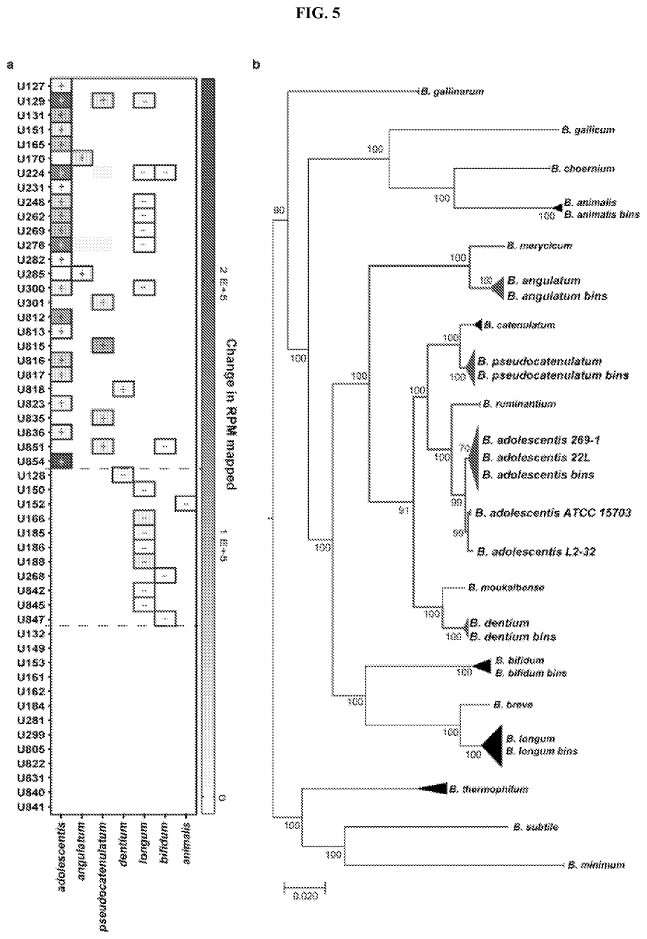

The pil^{RS} locus and bifidobacterial blooms We also explored the metagenomes of 51 participants who supplemented their diets with RSP for evidence of pil^{RS} loci. Metagenomic reads from each individual before and during RSP consumption were assembled into bins that could be associated with bacterial species. In total, 48 bifidobacterial bins were assembled from 38 of the metagenomes (Table 2); metagenomes from 13 participants lacked any bins classified as bifidobacteria. Bins from 11 metagenomes (assigned to B. dentium, B. longum, B. bifidum or B. animalis) did not contain homologs of the four pil^{RS} genes and none of them increased in abundance during RSP supplementation. The remaining 27 metagenomes contained bins assigned to B. adolescentis, B. angulatum, B. pseudocatenulatum or B. dentium (FIG. 5). In each of these metagenomes at least one bin contained a homologous pil^{RS} locus that was syntenous to the pil^{RS} genes in B. adolescentis 269-1. All are associated with the previously described “B. adolescentis” clade³⁶ (FIG. 5b). In 26 of 27 metagenomes containing the pil^{RS} locus, the reads mapped per million reads (RPM) in the bin containing the pil^{RS} genes increased between 117% and 9,730% during RSP consumption. Although not all pil^{RS}-containing bifidobacteria increased dramatically, bifidobacteria with the pil^{RS} locus accounted for the largest responses (FIG. 5a). Thus, a pil^{RS}-like tip pilin appears to be important—though not sufficient—for in vivo blooms, rapid increases in the size of bifidobacterial populations.

TABLE 2
CheckM summary of bin quality based on completeness and contamination for metagenomic bins from the human cohort study.

| | | | | # | | | | | | | Com- | Contam- |
|---|---|---|---|---|---|---|---|---|---|---|---|---|
| Metagenomic | Marker | # | # | Marker | | Single | Duplicated | | | | pleteness | ination |
| bin | Lineage | Genomes | Markers | Sets | Missing | copy | 2 | 3 | 4 | 5+ | (%) | (%) |
| B. adolescentis bin U127 | f_Bifidobacteriaceae | 77 | 464 | 220 | 0 | 461 | 3 | 0 | 0 | 0 | 100 | 0.76 |
| B. adolescentis bin U129 | f_Bifidobacteriaceae | 77 | 464 | 220 | 0 | 462 | 2 | 0 | 0 | 0 | 100 | 0.23 |
| B. adolescentis bin U131 | f_Bifidobacteriaceae | 77 | 464 | 220 | 0 | 462 | 2 | 0 | 0 | 0 | 100 | 0.61 |
| B. adolescentis bin U151 | f_Bifidobacteriaceae | 77 | 464 | 220 | 1 | 459 | 4 | 0 | 0 | 0 | 99.89 | 1.14 |
| B. adolescentis bin U165 | f_Bifidobacteriaceae | 77 | 464 | 220 | 0 | 464 | 0 | 0 | 0 | 0 | 100 | 0 |
| B. adolescentis bin U224 | f_Bifidobacteriaceae | 77 | 464 | 220 | 2 | 455 | 7 | 0 | 0 | 0 | 99.7 | 2.73 |
| B. adolescentis bin U231 | f_Bifidobacteriaceae | 77 | 464 | 220 | 33 | 422 | 9 | 0 | 0 | 0 | 94.1 | 2.54 |
| B. adolescentis bin U248 | f_Bifidobacteriaceae | 77 | 464 | 220 | 27 | 435 | 2 | 0 | 0 | 0 | 94.09 | 0.68 |
| B. adolescentis bin U262 | f_Bifidobacteriaceae | 77 | 464 | 220 | 7 | 456 | 1 | 0 | 0 | 0 | 97.73 | 0.45 |
| B. adolescentis bin U269 | f_Bifidobacteriaceae | 77 | 464 | 220 | 1 | 463 | 0 | 0 | 0 | 0 | 99.55 | 0 |
| B. adolescentis bin U276 | f_Bifidobacteriaceae | 77 | 464 | 220 | 5 | 459 | 0 | 0 | 0 | 0 | 99.11 | 0 |
| B. adolescentis bin U282 | f_Bifidobacteriaceae | 77 | 464 | 220 | 54 | 407 | 3 | 0 | 0 | 0 | 86.94 | 0.66 |
| B. adolescentis bin U300 | f_Bifidobacteriaceae | 77 | 464 | 220 | 1 | 462 | 1 | 0 | 0 | 0 | 99.85 | 0.23 |
| B. adolescentis bin U812 | f_Bifidobacteriaceae | 77 | 464 | 220 | 152 | 311 | 1 | 0 | 0 | 0 | 67.27 | 0.45 |
| B. adolescentis bin U813 | f_Bifidobacteriaceae | 77 | 464 | 220 | 40 | 416 | 8 | 0 | 0 | 0 | 92.15 | 1.64 |
| B. adolescentis bin U816 | f_Bifidobacteriaceae | 77 | 464 | 220 | 0 | 459 | 5 | 0 | 0 | 0 | 100 | 1.32 |
| B. adolescentis bin U817 | f_Bifidobacteriaceae | 77 | 464 | 220 | 18 | 446 | 0 | 0 | 0 | 0 | 97.41 | 0 |
| B. adolescentis bin U823 | f_Bifidobacteriaceae | 77 | 464 | 220 | 0 | 464 | 0 | 0 | 0 | 0 | 100 | 0 |
| B. adolescentis bin U836 | f_Bifidobacteriaceae | 77 | 464 | 220 | 12 | 446 | 6 | 0 | 0 | 0 | 98.18 | 1.97 |
| B. adolescentis bin U854 | f_Bifidobacteriaceae | 77 | 464 | 220 | 0 | 460 | 4 | 0 | 0 | 0 | 100 | 1.21 |
| B. angulatum bin U170 | f_Bifidobacteriaceae | 65 | 476 | 217 | 2 | 473 | 1 | 0 | 0 | 0 | 99.77 | 0.09 |
| B. angulatum bin U285 | f_Bifidobacteriaceae | 65 | 476 | 217 | 338 | 138 | 0 | 0 | 0 | 0 | 31.26 | 0 |
| B. animalis bin U152 | f_Bifidobacteriaceae | 70 | 502 | 225 | 7 | 493 | 2 | 0 | 0 | 0 | 98.71 | 0.28 |
| B. biJidum bin U224 | f_Bifidobacteriaceae | 65 | 476 | 217 | 63 | 413 | 0 | 0 | 0 | 0 | 89 | 0 |
| B. biJidum bin U268 | f_Bifidobacteriaceae | 65 | 476 | 217 | 89 | 384 | 3 | 0 | 0 | 0 | 83.34 | 0.75 |
| B. biJidum bin U847 | f_Bifidobacteriaceae | 65 | 476 | 217 | 4 | 465 | 7 | 0 | 0 | 0 | 98.16 | 1.17 |
| B. biJidum bin U851 | f_Bifidobacteriaceae | 65 | 476 | 217 | 6 | 459 | 11 | 0 | 0 | 0 | 97.7 | 2.14 |
| B. dentium bin U128 | f_Bifidobacteriaceae | 77 | 464 | 220 | 0 | 461 | 3 | 0 | 0 | 0 | 100 | 1.02 |
| B. dentium bin U818 | f_Bifidobacteriaceae | 77 | 464 | 220 | 3 | 446 | 11 | 4 | 0 | 0 | 99.34 | 2.92 |
| B. longum bin U129 | f_Bifidobacteriaceae | 65 | 476 | 217 | 21 | 447 | 5 | 3 | 0 | 0 | 96.93 | 3.05 |
| B. longum bin U150 | f_Bifidobacteriaceae | 65 | 476 | 217 | 91 | 381 | 4 | 0 | 0 | 0 | 83.54 | 1.38 |
| B. longum bin U166 | f_Bifidobacteriaceae | 65 | 476 | 217 | 0 | 475 | 1 | 0 | 0 | 0 | 100 | 0.46 |
| B. longum bin U185 | f_Bifidobacteriaceae | 65 | 476 | 217 | 1 | 471 | 4 | 0 | 0 | 0 | 99.54 | 0.81 |
| B. longum bin U186 | f_Bifidobacteriaceae | 65 | 476 | 217 | 0 | 476 | 0 | 0 | 0 | 0 | 100 | 0 |
| B. longum bin U188 | f_Bifidobacteriaceae | 65 | 476 | 217 | 0 | 476 | 0 | 0 | 0 | 0 | 100 | 0 |
| B. longum bin U224 | f_Bifidobacteriaceae | 65 | 476 | 217 | 132 | 340 | 4 | 0 | 0 | 0 | 74.64 | 0.88 |
| B. longum bin U248 | f_Bifidobacteriaceae | 65 | 476 | 217 | 179 | 287 | 9 | 1 | 0 | 0 | 67.14 | 1.96 |
| B. longum bin U262 | f_Bifidobacteriaceae | 65 | 476 | 217 | 67 | 406 | 3 | 0 | 0 | 0 | 87.05 | 0.92 |
| B. longum bin U269 | f_Bifidobacteriaceae | 65 | 476 | 217 | 61 | 407 | 6 | 2 | 0 | 0 | 91.07 | 1.06 |
| B. longum bin U276 | f_Bifidobacteriaceae | 65 | 476 | 217 | 125 | 350 | 1 | 0 | 0 | 0 | 72.38 | 0.12 |
| B. longum bin U300 | f_Bifidobacteriaceae | 65 | 476 | 217 | 96 | 333 | 47 | 0 | 0 | 0 | 82.51 | 11.85 |
| B. longum bin U842 | f_Bifidobacteriaceae | 65 | 476 | 217 | 4 | 465 | 7 | 0 | 0 | 0 | 98.16 | 1.17 |
| B. longum bin U845 | f_Bifidobacteriaceae | 65 | 476 | 217 | 3 | 458 | 12 | 3 | 0 | 0 | 99.02 | 2.61 |
| B. pseudocatenulatum bin | f_Bifidobacteriaceae | 77 | 464 | 220 | 53 | 410 | 1 | 0 | 0 | 0 | 85.4 | 1.72 |
| U129 | | | | | | | | | | | | |
| B. pseudocatenulatum bin | f_Bifidobacteriaceae | 77 | 464 | 220 | 46 | 412 | 6 | 0 | 0 | 0 | 86.9 | 1.29 |
| U301 | | | | | | | | | | | | |
| B. pseudocatenulatum bin | f_Bifidobacteriaceae | 77 | 464 | 220 | 0 | 462 | 2 | 0 | 0 | 0 | 100 | 0.68 |
| U815 | | | | | | | | | | | | |
| B. pseudocatenulatum bin | f_Bifidobacteriaceae | 77 | 464 | 220 | 0 | 463 | 1 | 0 | 0 | 0 | 100 | 0.23 |
| U835 | | | | | | | | | | | | |
| B. pseudocatenulatum bin | f_Bifidobacteriaceae | 77 | 464 | 220 | 137 | 326 | 1 | 0 | 0 | 0 | 70.83 | 0.23 |
| U851 | | | | | | | | | | | | |

### Example 5

### Pil^{RS} Loci in Human Gut Microbiomes

In the human cohort studies, there was no apparent relationship between the presence of the pil^{RS} locus with sex or ethnicity of participants (Table 3). In addition to the human cohort, we leveraged publicly available metagenome databases to assess if the pil^{RS} tip pilin is widespread in the human population. Specifically, the MGnify37 human gastrointestinal metagenome database was searched for matches to the tip pilin protein sequence. We found matches with high confidence (E-value <1e-300) in 72 metagenomes that were distributed across 9 countries on 4 continents, suggesting that this pil^{RS} tip pilin is indeed a widespread feature of the human gut microbiome. In the HumGut38 collection of human gut associated genomes and metagenomes, syntenic pil^{RS} loci were associated only with Bifidobacterium genomes.

TABLE 3
Participant sex and ethnicity data for metagenomic samples
with respect to the presence or absence of the pil^{RS} locus.

pil^{RS} locus

| | Present | Absent |
|---|---|---|
| Ethnicity | | |
| Caucasian/white | 14 | 19 |
| Latinx or Hispanic | 3 | 1 |
| Asian or Pacific Islander or | 7 | 4 |
| Hawaiian | | |
| Middle Eastern/North African | 1 | 0 |
| (MENA) | | |
| Unspecific multiethnic or other | 2 | 0 |
| Sex | | |
| Male | 8 | 7 |
| Female | 19 | 17 |

### Example 6

### Exemplary Synbiotic Composition

B. faecale 269-1 Potato Starch Adhesion Protocol

### Materials:

5 grams GS62 potato starch
YFCA broth with 0.1 M phosphate buffer at pH 7.0
B. faecale 269_1
15 mL screw-top tubes
Test tube roller
Anaerobic chamber

### Procedure:

Carried out in an anaerobic chamber.
1. Aliquot 10 mL of YCFA into the test tube and inoculate with 269_1.
2. Grow 269_1 at 37° C. on the test tube roller until the culture reaches an OD of 1 at 600 nm.
3. Aliquot 1 mL of the culture into each of 10 1.5 mL microcentrifuge tubes.
4. Centrifuge at 4000 rpm for 3 minutes.
5. Discard supernatant and retain pellet.
6. Wash each pellet twice with 1 mL of 0.1 M phosphate buffer (pH 7.0).
7. Resuspend each pellet with 1.1 mL of 0.1 M phosphate buffer (pH 7.0).
8. Transfer all of the suspensions into a 15 mL screw-top tube.
9. Add 5 grams of potato starch and mix well.
10. Allow tube to stand at room temperature for 1 hour, allowing the bacteria to attach to the potato starch and then sink to the bottom creating a bacteria/potato starch sediment.
11. Carefully remove the supernatant, trying to not disturb the sediment.
12. Cap the screw-top tube and move to −80° C. freezer until well-frozen.
13. Set the lyophilizer ballast to high vacuum.
14. Place the froze potato starch/bacteria into the lyophilizer and make sure that the valve on the top of the lyophilizer chamber is closed.
15. To run the Auto Mode press the panel switch labeled REFRIGERATION AUTO. The green LED above the switch will illuminate. This will start the refrigeration system. When the collector reaches −40° C., the vacuum pump will start. The Temperature and Vacuum Graph will indicate collector temperature and system vacuum. The LCD display will show the actual temperature of the collector. When the vacuum in the system is above 20 mBar the vacuum display will indicate “HI.” At 20 mBar and below, the display will show the actual vacuum. When the system vacuum is between 0.450 and 0.133 mBar, the lower green vacuum graph LED will flash.
16. Allow the lyophilizer to run for 6 hours and then check the samples. The amount of time to lyophilize depends on the amount of liquid that was removed before freezing. 6 hours should be sufficient if a majority of the liquid was removed. Fully dried, the potato starch/bacteria mixture will be a very fine, easy-flowing powder.

### LITERATURE CITED

1. Mohajeri, M. H. et al. The role of the microbiome for human health: from basic science to clinical applications. Eur J Nutr 57, 1-14 (2018).
2. Bendiks, Z. A., Knudsen, K. E. B., Keenan, M. J. & Marco, M. L. Conserved and variable responses of the gut microbiome to resistant starch type 2. Nutr Res 77, 12-28 (2020).
3. Millen, B. E. Scientific Report of the 2015 Dietary Guidelines Advisory Committee: United States Department of Agriculture and the Department of Health and Human Services; 2015.
4. Slavin, J. L. Position of the American Dietetic Association: health implications of dietary fiber. Journal of the American Dietetic Association 108, 1716-1731 (2008).
5. Sanna, S. et al. Causal relationships among the gut microbiome, short-chain fatty acids and metabolic diseases. Nature genetics 51, 600-605 (2019).
6. Baxter, N. T. et al. Dynamics of human gut microbiota and short-chain fatty acids in response to dietary interventions with three fermentable fibers. mBio 10 (2019).
7. Champ, M., Langkilde, A. M., Brouns, F., Kettlitz, B. & Bail-Collet, Y. L. Advances in dietary fibre characterisation. 2. Consumption, chemistry, physiology and measurement of resistant starch; implications for health and food labelling. Nutr Res Rev 16, 143-161 (2003).
8. Belenguer, A. et al. Two routes of metabolic cross-feeding between Bifidobacterium adolescentis and butyrate-producing anaerobes from the human gut. Appl Environ Microbiol 72, 3593-3599 (2006).
9. van der Hee, B. & Wells, J. M. Microbial regulation of host physiology by short-chain fatty acids. Trends Microbiol 29, 700-712 (2021).
10. Campos-Perez, W. & Martinez-Lopez, E. Effects of short chain fatty acids on metabolic and inflammatory processes in human health. Biochim Biophys Acta Mol Cell Biol Lipids 1866, 158900 (2021).
11. Abs, E., Leman, H. & Ferrière, R. A multi-scale eco-evolutionary model of cooperation reveals how microbial adaptation influences soil decomposition. Communications Biology 3, 520 (2020).
12. West, S. A., Griffin, A. S., Gardner, A. & Diggle, S. P. Social evolution theory for microorganisms. Nature Reviews Microbiology 4, 597-607 (2006).
13. Crouch, L. I. et al. Prominent members of the human gut microbiota express endo-acting O-glycanases to initiate mucin breakdown. Nature Communications 11, 4017 (2020).
14. Mukhopadhya, I. et al. Sporulation capability and amylosome conservation among diverse human colonic and rumen isolates of the keystone starch-degrader Ruminococcus bromii. Environ Microbiol 20, 324-336 (2018).
15. Shoham, Y., Lamed, R. & Bayer, E. A. The cellulosome concept as an efficient microbial strategy for the degradation of insoluble polysaccharides. Trends Microbiol 7, 275-281 (1999).
16. Venkataraman, A. et al. Variable responses of human microbiomes to dietary supplementation with resistant starch. Microbiome 4 (2016).
17. Ze, X., Duncan, S. H., Louis, P. & Flint, H. J. Ruminococcus bromii is a keystone species for the degradation of resistant starch in the human colon. ISME J 6, 1535-1543 (2012).
18. Duranti, S. et al. Genomic characterization and transcriptional studies of the starch-utilizing strain Bifidobacterium adolescentis 22L. Applied and Environmental Microbiology 80, 6080-6090 (2014).
19. Jung, D. H. et al. Complete genome sequence of Bifidobacterium adolescentis P2P3, a human gut bacterium possessing strong resistant starch-degrading activity. 3 Biotech 10, 31 (2020).
20. Katoh, T. et al. Enzymatic adaptation of Bifidobacterium bifidum to host glycans, viewed from glycoside hydrolyases and carbohydrate-binding modules. Microorganisms 8, 481 (2020).
21. Foroni, E. et al. Genetic analysis and morphological identification of pilus-like structures in members of the genus Bifidobacterium. Microb Cell Fact 10 Suppl 1, S16-S16 (2011).
22. Turroni, F. et al. Role of sortase-dependent pili of Bifidobacterium bifidum PRL2010 in modulating bacterium-host interactions. Proc Natl Acad Sci USA 110, 11151-11156 (2013).
23. Milani, C. et al. Bifidobacteria exhibit social behavior through carbohydrate resource sharing in the gut. Sci Rep 5, 15782 (2015).
24. Ciufo, S. et al. Using average nucleotide identity to improve taxonomic assignments in prokaryotic genomes at the NCBI. Int J Syst Evol Microbiol 68, 2386-2392 (2018).
25. O'Connell Motherway, M. et al. Characterization of ApuB, an extracellular type II amylopullulanase from Bifidobacterium breve UCC2003. Applied and Environmental Microbiology 74, 6271-6279 (2008).
26. Zhang, H. et al. dbCAN2: a meta server for automated carbohydrate-active enzyme annotation. Nucleic Acids Research 46, W95-W101 (2018).
27. Jones, P. et al. InterProScan 5: genome-scale protein function classification. Bioinformatics 30, 1236-1240 (2014).
28. Tester, R. F., Karkalas, J. & Qi, X. Starch-composition, fine structure and architecture. Journal of Cereal Science 39, 151-165 (2004).
29. Gallant, D. J., Bouchet, B., Buleon, A. & Perez, S. Physical characteristics of starch granules and susceptibility to enzymatic degradation. Eur J Clin Nutr 46 Suppl 2, S3-16 (1992).
30. Zhang, L. et al. Starch flocculation by the sweet potato sour liquid Is mediated by the adhesion of lactic acid bacteria to starch. Frontiers in Microbiology 8 (2017).
31. Wang, X. et al. A new Leuconostoc citreum strain discovered in the traditional sweet potato sour liquid fermentation as a novel bioflocculant for highly efficient starch production. Food Research International 144, 110327 (2021).
32. Jung, D. H. et al. Bifidobacterium adolescentis P2P3, a human gut bacterium having strong non-gelatinized resistant starch-degrading activity. J Microbiol Biotechnol 29, 1904-1915 (2019).
33. Szabo, L. et al. Structure of a family 15 carbohydrate-binding module in complex with xylopentaose. Evidence that xylan binds in an approximate 3-fold helical conformation. J Biol Chem 276, 49061-49065 (2001).
34. Stephen, P., Cheng, K. C. & Lyu, P. C. Crystal structure of circular permuted RoCBM21 (CP90): dimerisation and proximity of binding sites. PLoS One 7, e50488 (2012).
35. Cockburn, D. W. et al. Novel carbohydrate binding modules in the surface anchored α-amylase of Eubacterium rectale provide a molecular rationale for the range of starches used by this organism in the human gut. Mol Microbiol 107, 249-264 (2018).
36. Duranti, S., Longhi, G., Ventura, M., van Sinderen, D. & Turroni, F. Exploring the ecology of bifidobacteria and their genetic adaptation to the mammalian gut. Microorganisms 9, 8 (2021).
37. Mitchell, A. L. et al. MGnify: the microbiome analysis resource in 2020. Nucleic Acids Research 48, D570-D578 (2019).
38. Hiseni, P., Rudi, K., Wilson, R. C., Hegge, F. T. & Snipen, L. HumGut: a comprehensive human gut prokaryotic genomes collection filtered by metagenome data. Microbiome 9, 165 (2021).
39. Artzi, L., Bayer, E. A. & Morais, S. Cellulosomes: bacterial nanomachines for dismantling plant polysaccharides. Nature Reviews Microbiology 15, 83-95 (2017).
40. Bertoft, E. Chapter 2—Analyzing Starch Molecular Structure. In: Sjöö, M. & Nilsson, L. (eds). Starch in Food (Second Edition). Woodhead Publishing, 2018, pp 97-149.
41. Jung, D. et al. The presence of resistant starch-degrading amylases in Bifidobacterium adolescentis of the human gut. International Journal of Biological Macromolecules (2020).

### LITERATURE CITED—METHODS

1. McCleary, B. V. & Monaghan, D. A. Measurement of Resistant Starch. Journal of AOAC INTERNATIONAL 85, 665-675 (2019).
2. Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl Environ Microbiol 79, 5112-5120 (2013).
3. Schloss, P. D. et al. Introducing mothur: open-source, platform-independent, community-supported software for describing and comparing microbial communities. Appl Environ Microbiol 75, 7537-7541 (2009).
4. Lopez-Siles, M. et al. Cultured representatives of two major phylogroups of human colonic Faecalibacterium prausnitzii can utilize pectin, uronic acids, and host-derived substrates for growth. Appl Environ Microbiol 78, 420-428 (2012).
5. Bankevich, A. et al. SPAdes: a new genome assembly algorithm and its applications to single-cell sequencing. J Comput Biol 19, 455-477 (2012).
6. Aziz, R. K. et al. The RAST Server: Rapid Annotations using Subsystems Technology. BMC Genomics 9, 75 (2008).
7. Arkin, A. P. et al. KBase: The United States Department of Energy Systems Biology Knowledgebase. Nature Biotechnology 36, 566-569 (2018).
8. Wick, R. R., Judd, L. M., Gorrie, C. L. & Holt, K. E. Unicycler: Resolving bacterial genome assemblies from short and long sequencing reads. PLoS Comput Biol 13, e1005595 (2017).
9. Antipov, D., Korobeynikov, A., McLean, J. S. & Pevzner, P. A. hybridSPAdes: an algorithm for hybrid assembly of short and long reads. Bioinformatics 32, 1009-1015 (2016).
10. Duranti, S. et al. Genomic characterization and transcriptional studies of the starch-utilizing strain Bifidobacterium adolescentis 22L. Applied and Environmental Microbiology 80, 6080-6090 (2014).
11. Camacho, C. et al. BLAST+: architecture and applications. BMC Bioinformatics 10, 421 (2009).
12. Thompson, J. D., Higgins, D. G. & Gibson, T. J. CLUSTAL W: improving the sensitivity of progressive multiple sequence alignment through sequence weighting, position-specific gap penalties and weight matrix choice. Nucleic Acids Res 22, 4673-4680 (1994).
13. Kumar, S., Stecher, G. & Tamura, K. MEGA7: Molecular Evolutionary Genetics Analysis Version 7.0 for Bigger Datasets. Mol Biol Evol 33, 1870-1874 (2016).
14. Zuckerkandl, E. & Pauling, L. Evolutionary Divergence and Convergence in Proteins. In: Bryson, V. & Vogel, H. J. (eds). Evolving Genes and Proteins. Academic Press, 1965, pp 97-166.
15. Cock, P. J., Gruning, B. A., Paszkiewicz, K. & Pritchard, L. Galaxy tools and workflows for sequence analysis with applications in molecular plant pathology. Peer J 1, e167 (2013).
16. Liu, W. et al. IBS: an illustrator for the presentation and visualization of biological sequences. Bioinformatics 31, 3359-3361 (2015).
17. Wang, J., Shoemaker, N. B., Wang, G. R. & Salyers, A. A. Characterization of a Bacteroides mobilizable transposon, NBU2, which carries a functional lincomycin resistance gene. J Bacteriol 182, 3559-3571 (2000).
18. Jung, D. H. et al. Bifidobacterium adolescentis P2P3, a human gut bacterium having strong non-gelatinized resistant starch-degrading activity. J Microbiol Biotechnol 29, 1904-1915 (2019).
19. Crittenden, R. et al. Adhesion of bifidobacteria to granular starch and its implications in probiotic technologies. Appl Environ Microbiol 67, 3469-3475 (2001).
20. Foley, M. H., Martens, E. C. & Koropatkin, N. M. SusE facilitates starch uptake independent of starch binding in B. thetaiotaomicron. Mol Microbiol 108, 551-566 (2018).
21. Cockburn, D. W. et al. Novel carbohydrate binding modules in the surface anchored α-amylase of Eubacterium rectale provide a molecular rationale for the range of starches used by this organism in the human gut. Mol Microbiol 107, 249-264 (2018).
22. Bolger, A. M., Lohse, M. & Usadel, B. Trimmomatic: a flexible trimmer for Illumina sequence data. Bioinformatics 30, 2114-2120 (2014).
23. Nurk, S., Meleshko, D., Korobeynikov, A. & Pevzner, P. A. metaSPAdes: a new versatile metagenomic assembler. Genome Res 27, 824-834 (2017).
24. Peng, Y., Leung, H. C., Yiu, S. M. & Chin, F. Y. IDBA-UD: a de novo assembler for single-cell and metagenomic sequencing data with highly uneven depth. Bioinformatics 28, 1420-1428 (2012).
25. Wu, Y. W., Simmons, B. A. & Singer, S. W. MaxBin 2.0: an automated binning algorithm to recover genomes from multiple metagenomic datasets. Bioinformatics 32, 605-607 (2016).
26. Alneberg, J. et al. Binning metagenomic contigs by coverage and composition. Nature Methods 11, 1144-1146 (2014).
27. Kang, D. D., Froula, J., Egan, R. & Wang, Z. MetaBAT, an efficient tool for accurately reconstructing single genomes from complex microbial communities. Peer J 3, e1165 (2015).
28. Sieber, C. M. K. et al. Recovery of genomes from metagenomes via a dereplication, aggregation and scoring strategy. Nature Microbiology 3, 836-843 (2018).
29. Parks, D. H., Imelfort, M., Skennerton, C. T., Hugenholtz, P. & Tyson, G. W. CheckM: assessing the quality of microbial genomes recovered from isolates, single cells, and metagenomes. Genome Res 25, 1043-1055 (2015).
30. Overbeek, R. et al. The SEED and the Rapid Annotation of microbial genomes using Subsystems Technology (RAST). Nucleic Acids Res 42, D206-214 (2014).
31. Brettin, T. et al. RASTtk: A modular and extensible implementation of the RAST algorithm for building custom annotation pipelines and annotating batches of genomes. Scientific Reports 5, 8365 (2015).
32. Chaumeil, P. A., Mussig, A. J., Hugenholtz, P. & Parks, D. H. GTDB-Tk: a toolkit to classify genomes with the Genome Taxonomy Database. Bioinformatics 36, 1925-1927 (2019).
33. Price, M. N., Dehal, P. S. & Arkin, A. P. FastTree 2—approximately maximum-likelihood trees for large alignments. PLoS One 5, e9490-e9490 (2010).

We claim:

1. A synbiotic composition comprising: a) a resistant starch; and b) a bacterium comprising a resistant-starch-binding pilus.

2. The composition of claim 1, wherein said bacterium is a Bifidobacterium species.

3. The composition of claim 2, wherein said bacterium is a B. adolescentis clade species.

4. The composition of claim 2, wherein said bacterium is a B. adolescentis, B. angulatum, B. pseudocatenulatum or B. dentium.

5. The composition of claim 1, wherein said resistant starch is mixed with said bacterium.

6. The composition of claim 1, wherein said composition comprises a food product.

7. The composition of claim 1, wherein said composition comprises a food.

8. The composition of claim 1, wherein said composition comprises a nutraceutical.

9. The composition of claim 1, wherein said composition comprises a capsule.

10. A method comprising: administering a resistant starch and a bacterium comprising a resistant-starch-binding pilus to a subject.

11. The method of claim 10, wherein said administering comprising administering a synbiotic composition comprising said resistant starch and said bacterium to said subject.

12. The method of claim 10, wherein said administering comprising administering a probiotic comprising said bacterium to said subject.

13. The method of claim 10, wherein said administering comprises administering a prebiotic comprising said resistant starch to said subject.

14. The method of claim 10, wherein said administering comprises administering a prebiotic comprising said resistant starch and a probiotic comprising said bacterium to said subject.

15. The method of claim 10, wherein said subject has graft versus host disease.

16. The method of claim 10, wherein said subject has undergone a bone marrow transplant.

17. The method of claim 10, wherein said subject has cancer.

18. The method of claim 10, wherein said subject has an inflammatory bowel disease.
